(19) United States
(12) Patent Application Publication
(10) Pub. No.: US 2017/0056555 A1
(43) Pub. Date: Mar. 2, 2017

(54) LIPOSOME FOR DELIVERING EXTRACELLULAR MATRIX

(71) Applicant: SOGANG UNIVERSITY RESEARCH FOUNDATION, Seoul (KR)

(72) Inventors: Kwan Woo SHIN, Seoul (KR); Keel Yong LEE, Seoul (KR); Tae Kyu AHN, Seoul (KR); Gi Yoong TAE, Gwangju (KR)

(21) Appl. No.: 15/131,485

(22) Filed: Apr. 18, 2016

(30) Foreign Application Priority Data
Aug. 28, 2015 (KR) 10-2015-0122068

(51) Int. Cl. A61L 27/36; C12N 5/071; C12N 5/09; A61L 27/50

(52) U.S. Cl. CPC A61L 27/3633; A61L 27/50; C12N 5/0686; C12N 5/0693; A61L 2300/62; C12N 2501/999

(57) ABSTRACT

The present disclosure provides a liposome for delivering an extracellular matrix, a method for promoting cell growth, and a method for preparing a liposome for delivering an extracellular matrix. According to the present disclosure, the liposome for delivering an extracellular matrix promotes cell attachment and growth, and through this matter, the liposome for delivering an extracellular matrix can be applied to cell or tissue regeneration.

### CROSS-REFERENCE TO RELATED APPLICATIONS

This application claims the benefit and priority of Korean Patent Application No. 10-2015-0122068, filed Aug. 28, 2015. The entire disclosure of the above application is incorporated herein by reference.

### FIELD

The present invention relates to a liposome for delivering an extracellular matrix.

### BACKGROUND

In biology, the extracellular matrix is mainly in charge of the structural support of animals. The extracellular matrix pertains to the connective tissue of an animal. The extracellular matrix is composed of the interstitial matrix and the basement membrane. The interstitial matrix fills the interstitial spaces. Gels of polysaccharides and fibrous proteins fill the interstitial space and help a buffer action of the extracellular matrix. The basement membrane is organized like thin paper, and the epithelial tissue is disposed thereon.

The components of the extracellular matrix are produced by corresponding cells, and are secreted into the extracellular matrix via exocytosis. The newly produced extracellular matrix is secreted and incorporated into the existing cellular matrix. The extracellular matrix is composed of an interlocking mesh of fibrous proteins and glycosaminolycans. The extracellular matrix is composed of proteoglycans, such as heparan sulfate, chondroitin sulfate, and keratin sulfate, non-proteoglycan polysaccharides such as hyaluronic acid, fibers such as collagen and elastin, fibronectin, and laminin.

A liposome is a spherical vesicle having at least one lipid bilayer. The liposome is used to deliver nutrients and pharmaceutical drugs. The liposome is biocompatible since it has a similar structure to the biological membrane, and can include hydrophilic drugs therein due to the structure of the closed double layer, and thus the liposome is widely used as a drug delivery system for delivering the hydrophilic drugs very effectively. However, the liposome cannot only be easily absorbed in the liver and spleen by the reticuloendothelial system after the administration into the body, but also has structural instability due to protein attachment and liposome aggregation in the blood, resulting in the leakage of inclusion drugs and causing side effects in normal cells. Therefore, research on the modification of the liposomal surface with various polymers in order to stabilize the liposomal structure is being actively developed (Seo, D. H. et al. Polymer (Korea) 2005, 29, 277. and Park, Y. J. et al. Polymer (Korea) 2004, 28, 502).

Throughout the entire specification, many papers and patent documents are referenced and their citations are represented. The disclosure of the cited papers and patent documents are entirely incorporated by reference into the present specification and the level of the technical field within which the present invention falls, and the details of the present invention are explained more clearly.

### SUMMARY

### Technical Problem

The present inventors endeavored to develop a liposome that is capable of promoting cell attachment and growth by delivering the extracellular matrix to cells. As a result, the present inventors verified that the extracellular matrix, which is bound to the liposomal surface including an anionic lipid, is delivered into cells to promote cell attachment and growth, and completed the present invention.

Accordingly, an aspect of the present invention is to provide a liposome for delivering an extracellular matrix.

Another aspect of the present invention is to provide a method for promoting the cell growth.

Other purposes and advantages of the present disclosure will become more obvious with the following detailed description of the invention, claims, and drawings.

### Technical Solution

In accordance with an aspect of the present invention, there is provided a liposome for delivering an extracellular matrix, the liposome including: (a) a phospholipid membrane having an anionic lipid and a neutral lipid, which are self-assembled; and (b) an extracellular matrix bound to the anionic lipid by ionic boding to be disposed on a surface of the anionic lipid.

The present inventors have endeavored to develop a liposome that is capable of promoting cell attachment and growth by delivering the extracellular matrix to cells. As a result, the present inventors verified that the extracellular matrix, which is bound to the liposomal surface including an anionic lipid, is delivered into cells to promote cell attachment and growth.

Here, one of the main characteristics of the present invention is that the phospholipid membrane constituting the liposome for delivering an extracellular matrix of the present invention includes an anionic lipid.

As used herein, the term “anionic lipid” refers to any amphiphilic lipid having at least one anionic charge in the range of pH 4.0 to pH 8.0. The anionic lipid includes any anionic lipid that is known to a person skilled in the art.

According to an embodiment of the present invention, the anionic lipid is at least one selected from the group consisting of dioleoyl phosphatidylserine (DOPS), dimyristoyl-phosphatidyl glycerol (DMPG), dipalmitoyl-phosphatidyl glycerol (DPPG), diethylenetriamine pentaacetic acid (DPTA), 1,4-dipalmitoyl-tartarate-2,3-diglutaric acid (DPTGA), 1,4-disteroyl-tartarate-2,3-disuccinic acid (DSTSA), 2-carboxyheptadecanoyl heptadecylamide (CHHDA), dimyristoylphosphatidylserin (DMPS), dipalmitoylphosphatidylserin (D PPS), palmitoyl-oleoylphosphatidylserin (POPS), dioleoylphosphatidylglycerol (DOPG), palmitoyl-oleoylphosphatidylglycerol (POPG), dimyristoylphosphatidic acid (DM PA), dipalmitoylphosphatidic acid (DPPA), dioleoylphosphatidic acid (DOPA), palmitoyl-oleoylphosphatidic acid (POPA), cetyl phosphate (CetylP), and cholesterol hemisuccinate (CHEMS).

According to another embodiment of the present invention, the anionic lipid is at least one selected from the group consisting of DOPS, DMPG, DPPG, DPTA, DPTGA, DSTSA, and CHHDA.

According to a specific embodiment of the present invention, the anionic lipid is DOPS.

The anionic lipid constituting the phospholipid membrane of the liposome for delivering an extracellular matrix of the present invention includes any anionic lipid that is known to a person skilled in the art.

The lipid constituting the phospholipid membrane of the liposome for delivering an extracellular matrix includes a neutral lipid in addition to the anionic lipid.

As used herein, the term “neutral lipid” refers to a lipid that is uncharged or has a zwitterion form in the range of pH 4.0 to pH 8.0. The neutral lipid includes any neutral lipid that is known to a person skilled in the art.

According to an embodiment of the present invention, the neutral lipid is at least one selected from the group consisting of 1,2-dioleoyl-sn-glycero-3-phosphocholine (DOPC), 1-palmitoyl-2-oleoyl-sn-glycero-3-phosphoethanolamine (POPE), cholesterol, 1,2-distearoyl-sn-glycero-3-phosphocholine (DSPC), 1,2-dipalmitoyl-sn-glycero-3-phosphocholine (DPPC), 1-palmitoyl-2-oleoyl-sn-glycero-3-phosphocholine (POPC), 1,2-dioleoyl-sn-glycero-3-phosphoethanolamine (DOPE), N-palmitoyl-D-erythro-sphingosylphosphorylcholine (SM), 1,2-dilauroyl-sn-glycero-3-phosphoethanolamine (DLPE), 2-diphytanoyl-sn-glycero-3-phosphoethanolamine (DiPPE), cholesterol, phosphatidyl choline, phosphatidyl ethanolamine, tetraether lipid, ceramide, sphigolipid, diacryl glycerol, and glyceride.

According to another embodiment of the present invention, the neutral lipid is at least one selected from the group consisting of DOPC, POPE, cholesterol, DSPC, DPPC, POPC, and DOPE.

According to a specific embodiment of the present invention, the neutral lipid is DOPC, POPE, and cholesterol.

The neutral lipid constituting the phospholipid membrane of the liposome for delivering an extracellular matrix of the present invention includes any neutral lipid that is known to a person skilled in the art.

The liposome for delivering an extracellular matrix of the present invention has a phospholipid membrane composed of an anionic lipid and a neutral lipid.

According to an embodiment of the present invention, the phospholipid membrane contains 1-30 mol % of the anionic lipid.

According to another embodiment of the present invention, the phospholipid membrane contains 1-25 mol %, 1-20 mol %, 5-25 mol %, or 5-20 mol % of the anionic lipid.

According to a specific embodiment of the present invention, the phospholipid membrane contains 10-20 mol % of the anionic lipid.

Here, another main characteristic of the present invention is that the extracellular matrix is bound to the surface of the liposome for delivering an extracellular matrix by ionic bonding with the anionic lipid constituting the phospholipid membrane of the liposome, and the polymerization reaction of the extracellular matrix leads to the self-assembly to induce an additional protein to the surface. Monomers are induced to the anionic lipid, and the induced monomers constitute a polymer through the self-assembly, a polymerization reaction. For example, in the case of collagen, monomers are induced to form a polymer through a polymerization. In the case of fibronectin, monomeric molecules are induced, and then the structure of the monomeric molecules is changed to unfold the folding structure thereof, and the unfolded fibronectin components are linked to a polymer.

According to an embodiment of the present invention, the extracellular matrix is at least one selected from the group consisting of fibronectin, collagen, laminin, elastin, integrin, and glycosaminoglycan.

According to another embodiment of the present invention, the extracellular matrix is at least one selected from the group consisting of fibronectin, collagen, laminin, and elastin.

According to a specific embodiment of the present invention, the extracellular matrix is at least one selected from the group consisting of fibronectin and collagen.

The liposome for delivering an extracellular matrix of the present invention contains 1-30 mol % of an anionic lipid, 70-99 mol % of a neutral lipid, and an extracellular matrix.

According to an embodiment of the present invention, the phospholipid membrane of the liposome for delivering an extracellular matrix is composed of DOPC, POPE, DOPS, and cholesterol.

According to another embodiment of the present invention, the phospholipid membrane contains 1-30 mol % of DOPS.

According to a specific embodiment of the present invention, the phospholipid membrane contains DOPC, POPE, DOPS, and cholesterol in 30-70 mol %, 1-30 mol %, 1-30 mol %, and 10-40 mol %, respectively.

The liposome for delivering an extracellular matrix of the present invention is a nano-sized liposome.

According to an embodiment of the present invention, the liposome for delivering an extracellular matrix has a size of 10-500 nm.

According to another embodiment of the present invention, the liposome for delivering an extracellular matrix has a size of 10-400 nm, 10-300 nm, 10-200 nm, or 50-150 nm.

According to another embodiment of the present invention, the present invention provides a pharmaceutical composition, containing a pharmaceutically effective amount of the liposome for delivering an extracellular matrix and a pharmaceutically acceptable carrier, for cell or tissue regeneration.

The present invention may be provided in the form of a pharmaceutical composition, containing a pharmaceutically effective amount of the liposome for delivering an extracellular matrix of the present invention and a pharmaceutically acceptable carrier, for cell or tissue regeneration. As used herein, the term “pharmaceutically effective amount” refers to a sufficient amount of the above-described liposome for delivering an extracellular matrix to attain cell or tissue regeneration efficacy. The pharmaceutical composition of the present invention contains a pharmaceutically acceptable carrier, in addition to the effective gradient compound.

The pharmaceutically acceptable carrier contained in the pharmaceutical composition of the present invention is usually used at the time of formulation, and examples thereof may include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil. The pharmaceutical composition of the present invention may further contain a lubricant, a wetting agent, a sweetening agent, a flavoring agent, an emulsifier, a suspending agent, a preservative, and the like, in addition to the above ingredient. Suitable pharmaceutically acceptable carriers and agents are described in detail in Remington's Pharmaceutical Sciences (19th ed., 1995).

A suitable dose of the pharmaceutical composition of the present invention may vary depending on various factors, such as the method for formulation, manner of administration, the age, body weight, gender, and morbidity of the patient, diet, food, time of administration, route of administration, excretion rate, and response sensitivity.

Meanwhile, the dose of the pharmaceutical composition of the present invention is preferably 0.001 μg/kg to 100 mg/kg (body weight) per day.

The pharmaceutical composition of the present invention may be administered orally or parenterally, and examples of the parenteral administration may include transdermal patch, intravenous injection, subcutaneous injection, intramuscular injection, intraperitoneal injection, and transdermal injection.

The pharmaceutical composition of the present invention is formulated in the unit dosage form or into a multidose container using a pharmaceutically acceptable carrier and/or excipient according to the method that can be easily carried out by a person having an ordinary skill in the art to which the present invention pertains. Here, the dosage form may be a solution in an oily or aqueous medium, a suspension, an emulsion, an extract, a powder, granules, a tablet, or a capsule, and may further contain a dispersant or a stabilizer.

According to an embodiment of the present invention, the pharmaceutical composition of the present invention has a dosage form for external skin application.

The dosage form for external skin application is, but is not particularly limited to, a powder, gel, ointment, cream, liquid, or aerosol.

According to still another embodiment of the present invention, the present invention provides a cosmetic composition, containing a pharmaceutically effective amount of the liposome for delivering an extracellular matrix and a pharmaceutically acceptable carrier, for cell or tissue regeneration.

The present invention may be provided in the form of a cosmetic composition, containing cosmetically effective amount of the liposome for delivering an extracellular matrix of the present invention and a cosmetically acceptable carrier, for cell or tissue regeneration. As used herein, the term “cosmetically effective amount” refers to a sufficient amount of the above-described liposome for delivering an extracellular matrix to attain skin regeneration efficacy.

The cosmetic composition of the present invention contains a cosmetically acceptable carrier, in addition to the effective gradient compound.

The cosmetic composition of the present invention may be formulated into any dosage form that is conventionally prepared, and examples thereof may include a solution, a suspension, an emulsion, a paste, a gel, a cream, a lotion, a powder, a soap, a surfactant-containing cleansing, an oil, a powder foundation, an emulsion foundation, a wax foundation, and a spray, but are not limited thereto. More specifically, the cosmetic composition of the present invention may be prepared in the dosage form of an emollient lotion, nourishing lotion, nourishing cream, massage cream, essence, eye cream, cleansing cream, cleansing foam, cleansing water, pack, spray or powder.

In cases where the dosage form of the present invention is a paste, cream, or gel, examples of the carrier component may include an animal oil, a plant oil, wax, paraffin, starch, tracant, a cellulose derivative, polyethylene glycol, silicon, bentonite, silica, talc, or zinc oxide.

In cases where the dosage of the present invention is a powder or a spray, examples of the carrier component may include lactose, talc, silica, aluminum hydroxide, calcium silicate, or a polyamide powder. Especially, in cases where the dosage form of the present invention is a spray, the dosage form may additionally include a propellant, such as chlorofluorohydrocarbon, propane/butane, or dimethyl ether.

In cases where the dosage form of the present invention is a solution or an emulsion, examples of the carrier component may include a solvent, a solubilizer, or an emulsifier may be used as a carrier component: for example water, ethanol, isopropanol, ethyl carbonate, ethyl acetate, benzyl alcohol, benzyl benzoate, propylene glycol, 1,3-butyl glycol oil, glycerol aliphatic ester, polyethylene glycol, or fatty acid ester of sorbitan.

In cases where the dosage form of the present invention is a suspension, examples of the carrier component may include liquid diluents, such as water, ethanol, and propylene glycol; suspending agents, such as ethoxylated isostearyl alcohol, polyoxyethylene sorbitol ester, and polyoxyethylene sorbitan ester; microcrystalline cellulose; aluminum metahydroxide; bentonite; agar; and tragacanth.

In cases where the dosage form of the present invention is a surfactant-containing cleansing, examples of the carrier component may include aliphatic alcohol sulfate, aliphatic alcohol ether sulfate, sulfosuccinate monoester, isethionate, imidazolium derivatives, methyl taurate, sarcosinate, fatty acid amide ether sulfate, alkyl amido betaine, aliphatic alcohol, fatty acid glyceride, fatty acid diethanolamide, plant oil, lanoline derivatives, and ethoxylated glycerol fatty acid ester.

The components contained in the cosmetic composition of the present invention includes components that are usually used in the cosmetic composition, in addition to the active ingredient and the carrier component, and for example, may include common aids, such as an antioxidant, a stabilizer, a solubilizer, vitamins, a pigment, and a flavoring.

According to another embodiment of the present invention, the present invention provides a method for promoting cell growth, the method including a step of bringing the liposome for delivering an extracellular matrix into contact with cells.

According to an embodiment of the present invention, the liposome for delivering an extracellular matrix is co-incubated with animal cells to promote the growth of the cells.

The liposome for delivering an extracellular matrix of the present invention promotes cell attachment and growth, compared with a control.

In accordance with another aspect of the present invention, there is provided a method for preparing a liposome for delivering an extracellular matrix, the method including the steps of: (a) preparing an anionic liposome by dissolving an anionic lipid and a neutral lipid in an organic solvent; and (b) binding an extracellular matrix to a surface of the anionic liposome.

The method of the present invention is directed to a method for preparing the liposome for delivering an extracellular matrix, and thus the overlapping descriptions of the method of the present invention and the above-described liposome for delivering an extracellular matrix of the present invention, such as the components of the phospholipid membrane and the extracellular matrix and the composition of the phospholipid membrane, are omitted to avoid excessive complication of the specification due to repetitive descriptions thereof.

### Advantageous effects

Features and advantages of the present invention are summarized as follows:

(a) The present invention provides a liposome for delivering an extracellular matrix, a method for promoting cell growth, and a method for preparing a liposome for delivering an extracellular matrix.

(b) The present invention provides a method for promoting cell attachment and growth by delivering an extracellular matrix into cells through a liposome.

### BRIEF DESCRIPTION OF THE DRAWINGS

FIG. 1 shows sizes of extracellular matrix-induced liposomes.

FIG. 2 shows the results of the delivery of extracellular matrix-incorporating liposomes to Hela cells.

FIG. 3 shows fluorescent images of cell growth of Hela cells and HEK 293 cells treated with extracellular matrix-induced liposomes, compared with a control.

FIG. 4 shows attachment ratios of HeLa cells and HEK 293 cells treated with extracellular matrix-induced liposomes.

FIG. 5 shows observation results for 36 hours of growth procedures of HEK 293 cells treated with extracellular matrix-induced liposomes.

FIG. 6 shows observation results for 36 hours of growth procedures of HeLa cells treated with extracellular matrix-induced liposomes.

### DETAILED DESCRIPTION

Hereinafter, the present invention will be described in detail with reference to examples. These examples are only for illustrating the present invention more specifically, and it will be apparent to those skilled in the art that the scope of the present invention is not limited by these examples.

### EXAMPLES

Preparation of liposomes containing cellular matrix (collagen or fibronectin) and verification of extracellular matrix delivery

Induction of Extracellular Matrix Using Anionic Charges

The lipids constituting the cell membrane were purchased from Avanti lipid, and, as the cellular matrix, collagen was purchased from Sigma-Aldrich and fibronectin was purchased from Cytoskeleton, Inc.

First, in order to assemble the cellular membrane through self-assembly, the following lipid constitution including negatively charged 1,2-dioleoyl-sn-glycero-3-phospho-L-serine (DOPS) was selected to form anionic charges outside the cellular membrane. 1,2-dioleoyl-sn-glycero-3-ethylphosphocholine (DOPC): 1-palmitoyl-2-oleoyl-sn-glycero-3-phosphoethanolamine (POPE): 1,2-dioleoyl-sn-glycero-3-phospho-L-serine (DOPS): cholesterol (CHOL)=4:1:1:2 (mol). The lipids with the above ratio were allowed to react with chloroform (CHCl₃) at 1 mg lipid/ml, followed by coating on glass. Then, an organic solvent was removed using nitrogen, and the resultant material was left in a vacuum state for 1 hour in order to completely remove the residual organic solvent. Thereafter, the resultant material was allowed to react with 0.28 M sucrose and 2 mM 2-(N-morpholino)ethane sulfonic acid (MES) at pH 4.2 (in cases of collagen induction) or 0.28 M sucrose and 2 mM Tris-HCl at pH 7.4 (in cases of fibronectin induction). The synthetic liposome contains DOPS and thus has anionic charge, and induces ionic bonding with collagen or fibronectin using the anionic charge. Specifically, a 1 mg/ml collagen solution is denatured at 80° C., or dissolved in 0.05 M HCl, which is an acidic solution, to be prepared in the form of a monomeric molecule or a small fibril, followed by pretreatment. Since, at high pH, collagen is self-assembled before it is induced into the liposome, a collagen induction reaction was carried out at relatively low pH. The thickness of the collagen on the liposomal surface varies depending on the pretreatment method. The pre-treated collagen was dropped in a vesicle (bare liposome) solution, followed by reaction at 37° C. for 30 minutes, and then the pH of the resultant material was adjusted to pH of 7.4 using 0.28 M glucose and 0.01 mM KOH. 1 mg/ml of fibronectin was dissolved in PBS buffer, followed by reaction at 37° C. for 2 hours. At the time of reaction, the humidity was maintained at 99%, thereby preventing the occurrence of osmotic pressure between the outside and the inside of the liposome. For fluorescent tagging of the liposome, 1,2-dioleoyl-sn-glycero-3-phosphoethanolamine-N-(lissamine rhodamine B sulfonyl) (N-Rh-DOPE) was used. Fluorescein Isothiocyanate (FITC) fluorescent tagging was used for collagen, and HiLyte Fluor 488 (AnaSpec) was used for fibronectin.

Measurement of Size of Liposomes

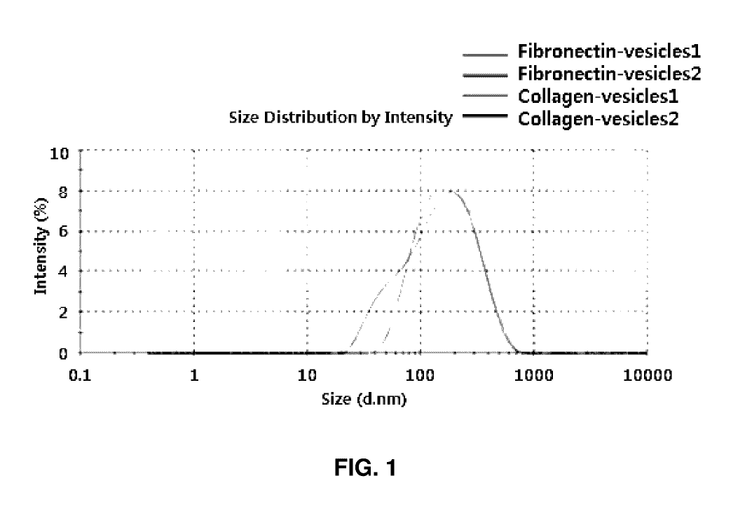

In order to measure the size of the prepared liposomes, a dynamic light scattering (DLS) apparatus was used. At the time of liposome synthesis, the size of the liposomes was controlled to 100 nm by using a polycarbonate filter, and an extracellular matrix was induced to the controlled liposomes, and then the size of the liposomes were measured through DSL. After the extracellular matrix was induced, the size of the liposomes was verified to increase to about 10 nm (FIG. 1).

Delivery to Actual Cells through Prepared Liposome

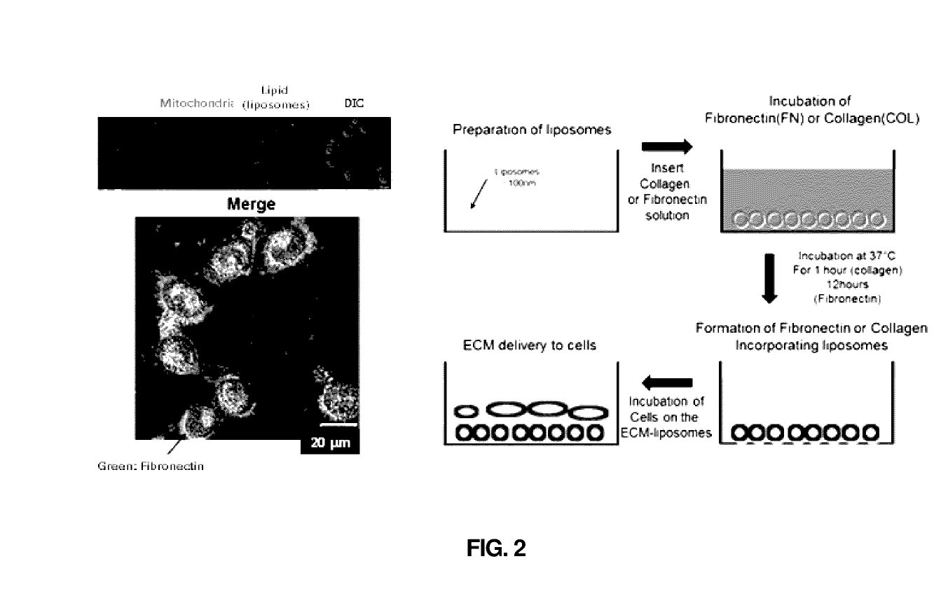

The delivery of the extracellular matrix was investigated by fluorescent tagging the prepared liposome and extracellular matrix and then incubating the liposome and extracellular matrix together with actual cells (FIG. 2). The incubation was carried out for 72 hours under incubation conditions of 37° C., 5% carbon dioxide, and 99% humidity in the medium composition of DMEM, 10% FBS, and 1% Penicillin-Streptomycin. The lipids move inside the cell by endocytosis, and the extracellular matrix is formed outside the cell. It was verified that when fibronectin incorporating liposomes and Hela cells were incubated at the same time, the cells react with the fibronectin incorporating liposomes to utilize the fibronectin, which is an extracellular matrix outside, as an extracellular matrix thereof, and the remaining lipids move into the cells.

### Verification of Cell Growth

Effect of Extracellular Matrix-Induced Liposomes on Growth of HeLa Cells and HEK 293 Cells

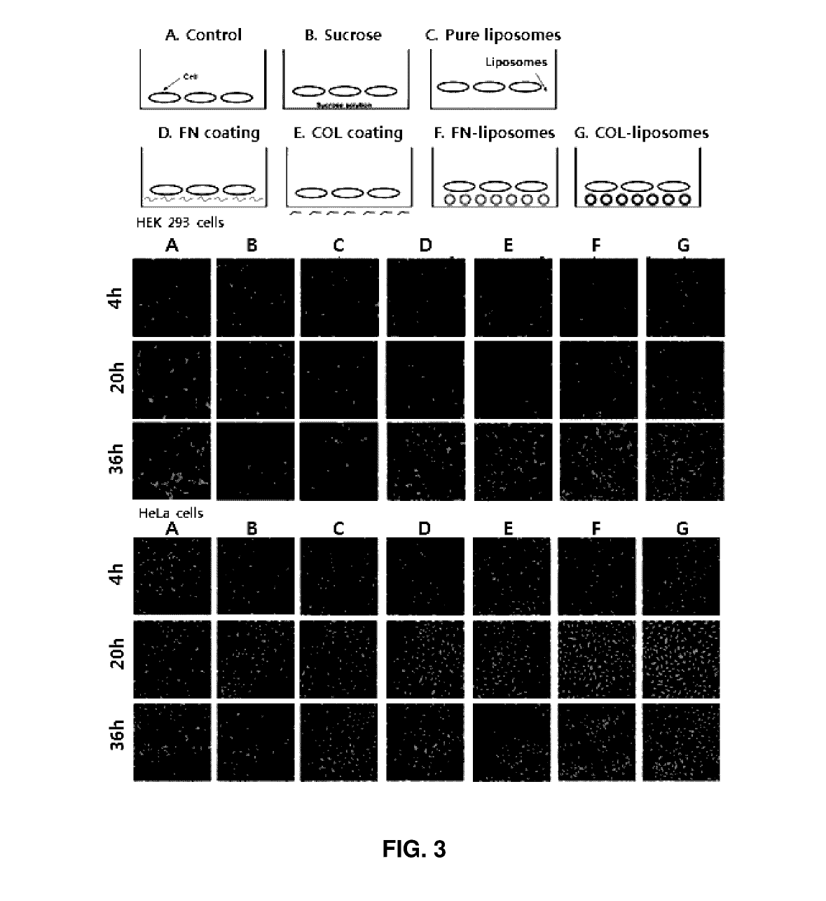

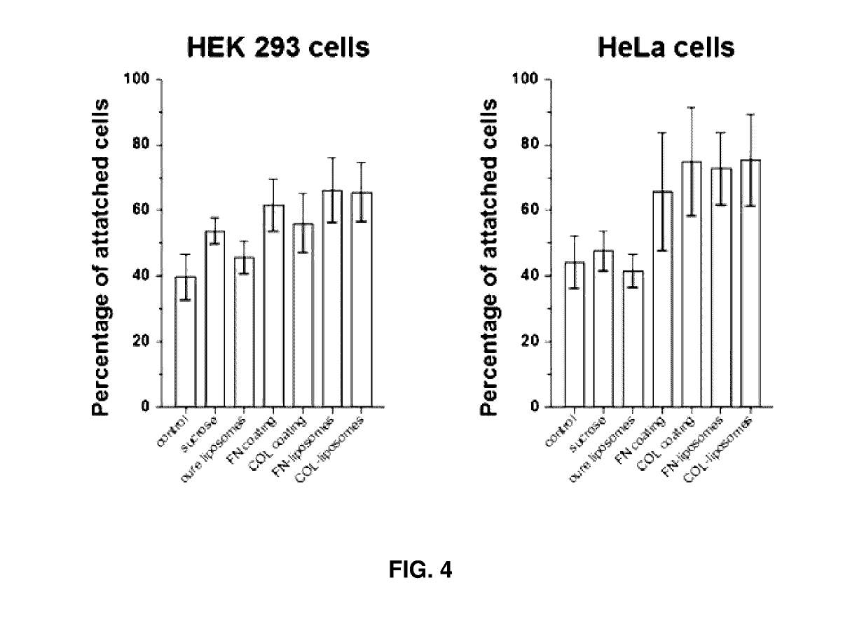

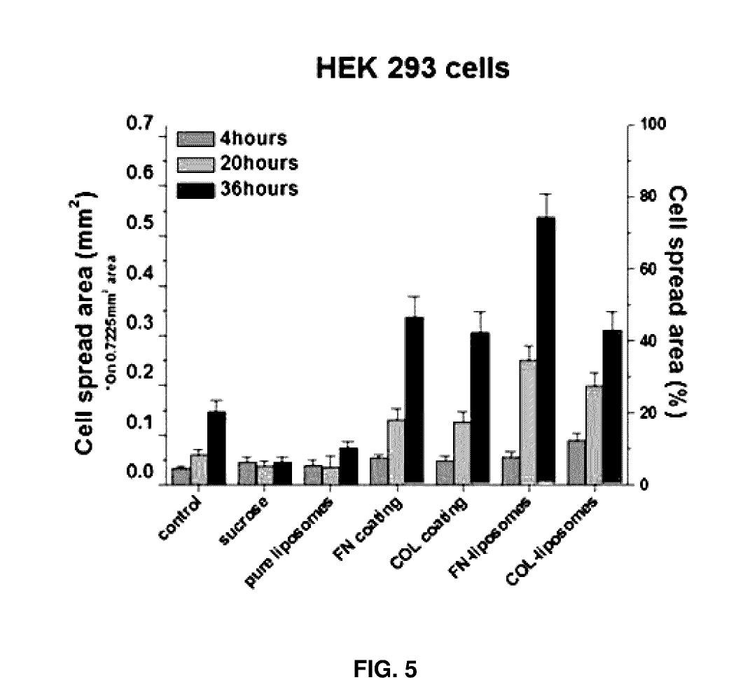

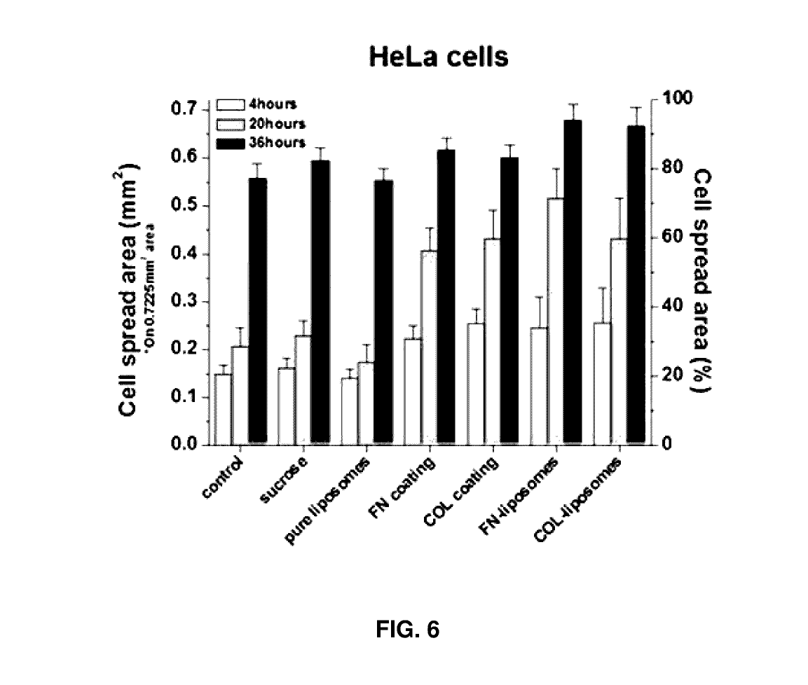

In order to investigate the effect of the prepared extracellular matrix on cell attachment and cell growth, the liposomes prepared in the present invention were compared and analyzed with a control. After Hela cells or HEK-293 cells were incubated for 3 hours in (a) a negative control, (b) 0.28 M sucrose 0.2 mM Tris-HCl pH 7.4, (c) pure liposomes, (d) fibronectin coating, (e) collagen coating, (f) FN-liposome, and (g) COL-liposome, the cell attachment was investigated and the cell growth condition was investigated at 6 hours, 20 hours, and 36 hours. It was verified through the results shown in FIGS. 3 to 6 that the induction of the extracellular matrix onto the cellular membrane was helpful in cell attachment and cell growth compared with the other cases. For verification of cell attachment, 105 cell seeds were incubated at 37° C. for 4 hours in Dulbecco's Modified Eagle Medium (DMEM) within each cell incubation flask. Then, the non-attached cells were removed by using PBS and the attached cells were counted to verify how many cells are left out of the existing 105 cells, and then the percentage of attached cells was calculated. Through five tests for each case, the standard deviation was calculated.

It was verified through FIG. 2 that, for the extracellular proteins existing outside the liposome, the lipid that has constituted existing vesicles entered the cells through the fusion of the vesicles and cells (blue in FIG. 2), and the extracellular matrix was delivered to the outside of the cells.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

What is claimed is:

1. A liposome for delivering an extracellular matrix, the liposome comprising:
(a) a phospholipid membrane having an anionic lipid and a neutral lipid, which are self-assembled; and
(b) an extracellular matrix bound to the anionic lipid by ionic boding to be disposed on a surface of the anionic lipid.

2. The liposome of claim 1, wherein the anionic lipid is at least one selected from the group consisting of dioleoyl phosphatidylserine (DOPS), dimyristoyl-phosphatidyl glycerol (DMPG), dipalmitoyl-phosphatidyl glycerol (DPPG), diethylenetriamine pentaacetic acid (DPTA), 1,4-dipalmitoyl-tartarate-2,3-diglutaric acid (DPTGA), 1,4-disteroyl-tartarate-2,3-disuccinic acid (DSTSA), 2-carboxyheptadecanoyl heptadecylamide (CHHDA), dimyristoylphosphatidylserin (DMPS), dipalmitoylphosphatidylserin (DPPS), palmitoyl-oleoylphosphatidylserin (POPS), dioleoylphosphatidylglycerol (DOPG), palmitoyl-oleoylphosphatidylglycerol (POPG), dimyristoylphosphatidic acid (DMPA), dipalmitoylphosphatidic acid (DPPA), dioleoylphosphatidic acid (DOPA), palmitoyl-oleoylphosphatidic acid (POPA), cetyl phosphate (CetylP), and cholesterol hemisuccinate (CHEMS).

3. The liposome of claim 1, wherein the neutral lipid is at least one selected from the group consisting of 1,2-dioleoyl-sn-glycero-3-phosphocholine (DOPC), 1-palmitoyl-2-oleoyl-sn-glycero-3-phosphoethanolamine (POPE), cholesterol, 1,2-distearoyl-sn-glycero-3-phosphocholine (DSPC), 1,2-dipalmitoyl-sn-glycero-3-phosphocholine (DPPC), 1-palmitoyl-2-oleoyl-sn-glycero-3-phosphocholine (POPC), 1,2-dioleoyl-sn-glycero-3-phosphoethanolamine (DOPE), N-palmitoyl-D-erythro-sphingosylphosphorylcholine (SM), 1,2-dilauroyl-sn-glycero-3-phosphoethanolamine (DLPE), 2-diphytanoyl-sn-glycero-3-phosphoethanolamine (DiPPE), cholesterol, phosphatidyl choline, phosphatidyl ethanolamine, tetraether lipid, ceramide, sphigolipid, diacryl glycerol, and glyceride.

4. The liposome of claim 1, wherein the liposome comprises 1-30 mole % of the anionic lipid.

5. The liposome of claim 1, wherein the extracellular matrix is at least one selected from the group consisting of fibronectin, collagen, laminin, elastin, integrin, and glycosaminoglycan.

6. The liposome of claim 1, wherein the liposome is composed of DOPC: POPE: DOPS: cholesterol.

7. The liposome of claim 1, wherein the liposome has a size of 10 nm to 500 nm.

8. The liposome of claim 1, wherein the extracellular matrix is at least one selected from the group consisting of fibronectin, collagen, laminin, elastin, integrin, and glycosaminoglycan, wherein the liposome is composed of DOPC: POPE: DOPS:
cholesterol, and wherein the liposome has a size of 10 nm to 500 nm.

9. A pharmaceutical composition comprising a pharmaceutically effective amount of the liposome for delivering an extracellular matrix of claim 1, and a pharmaceutically acceptable carrier.

10. The pharmaceutical composition of claim 9, wherein the extracellular matrix is at least one selected from the group consisting of fibronectin, collagen, laminin, elastin, integrin, and glycosaminoglycan, wherein the liposome is composed of DOPC:
POPE: DOPS: cholesterol, and wherein the liposome has a size of 10 nm to 500 nm.

11. A cosmetic composition comprising a cosmetically effective amount of the liposome for delivering an extracellular matrix of claim 1, and a cosmetically acceptable carrier.

12. A method for promoting cell growth, the method comprising:
bringing the liposome for delivering an extracellular matrix of claim 1 into contact with animal cells.

13. A method for preparing a liposome for delivering an extracellular matrix, the method comprising:
(a) preparing an anionic liposome by dissolving an anionic lipid and a neutral lipid in an organic solvent; and
(b) binding an extracellular matrix to a surface of the anionic liposome.
